(19) United States
(12) Patent Application Publication
(10) Pub. No.: US 2021/0169343 A1
(43) Pub. Date: Jun. 10, 2021

(54) SYSTEM AND METHOD FOR DETECTING HOT FLASHES BASED ON HEART RATE PATTERNS

(71) Applicant: KONINKLIJKE PHILIPS N.V., Eindhoven (NL)

(72) Inventor: Alberto Giovanni BONOMI, Eindhoven (NL)

(21) Appl. No.: 17/115,864

(22) Filed: Dec. 9, 2020

Related U.S. Application Data
(60) Provisional application No. 62/945,918, filed on Dec. 10, 2019.

(51) Int. Cl. A61B 5/024; A61B 5/00

(52) U.S. Cl. CPC A61B 5/024; A61B 5/6801; A61B 5/7275; A61B 2560/0242; A61B 5/7264; A61B 2562/0219; A61B 5/7246

(57) ABSTRACT

A method of detecting the occurrence of a hot flash in an individual including obtaining heart rate sequence data for the individual for a predetermined period of time, wherein the heart rate sequence data is based on heartbeat data of the individual that is detected by a sensor unit worn by the individual, providing the heart rate sequence data to a computational model component, wherein the computational model component is structured and configured to examine the heart rate sequence data over time to determine a probability that the individual is experiencing a hot flash based on monitoring the heart rate sequence data for a pattern wherein heart rate decreases below a baseline range and then increases above the baseline range, and analyzing the heart rate sequence data in the computational model component to determine the probability.

### CROSS-REFERENCE TO PRIOR APPLICATIONS

This application claims the benefit of U.S. Provisional Application No. 62/945,918, filed on 10 Dec. 2019. This application is hereby incorporated by reference herein.

### BACKGROUND OF THE INVENTION

### 1. Field of the Invention

The disclosed concept pertains to a system and method for detecting the occurrence of hot flashes in individuals, and, in particular, to a system and method for detecting the occurrence of hot flashes in individuals based on heart rate patterns. In one aspect, if a hot flash is detected, therapeutic measures for the hot flash are automatically initiated.

### 2. Description of the Related Art

Hot flashes are sudden-onset, spontaneous and episodic sensations of warmth, usually felt on the chest, neck, and face, immediately followed by an outbreak of sweating. They are the most common reason that women seek medical care during the peri-menopausal period, especially if the symptoms impair quality of life. Frequency and severity of hot flashes can increase during the transition to menopause, and typically peak at approximately one year after the final menstrual period. Hot flashes can persist for six months to several years and, on average, they last less than five minutes. The average frequency varies from ten times per day to several times per week.

Since hot flashes cause heat and sweating in the chest area, they can be detected by monitoring galvanic skin response (GSR) at the chest. However, wearable sensors for measuring GSR at the chest are cumbersome, as they require adhesive material for skin attachment, which degrades over time and requires periodic maintenance.

There is thus a need for a convenient and low-cost solution to support long-term monitoring of hot flashes in women.

### SUMMARY OF THE INVENTION

Accordingly, it is an object of the present invention to provide, in one embodiment, a method of detecting an occurrence of a hot flash in an individual, the method including obtaining heart rate sequence data for the individual for a predetermined period of time, wherein the heart rate sequence data is based on heartbeat data of the individual that is detected by a sensor unit worn by the individual, providing the heart rate sequence data to a computational model component, wherein the computational model component is structured and configured to examine the heart rate sequence data over time to determine a probability that the individual is experiencing a hot flash based on monitoring the heart rate sequence data for a pattern wherein heart rate decreases below a baseline range and then increases above the baseline range, and analyzing the heart rate sequence data in the computational model component to determine the probability. The method may further include assessing the probability to determine whether a hot flash is indicated, and if a hot flash is indicated by the probability, causing an environmental parameter control apparatus associated with the individual to initiate therapeutic measures for the hot flash. In addition, determining the occurrence of hot flashes could be used to assess effectiveness of intervention strategies such as medication, diet and lifestyle factors aimed at reducing the likelihood or severity of hot flash events.

In another embodiment, system for detecting an occurrence of a hot flash in an individual is provided. The system includes a controller including a computational model component, wherein the computational model component is structured and configured to receive heart rate sequence data that is based on heartbeat data of the individual that is detected by a sensor worn by the individual and examine the heart rate sequence data over time to determine a probability that the individual is experiencing a hot flash based on monitoring the heart rate sequence data for a pattern wherein heart rate decreases below a baseline range and then increases above the baseline range.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1A shows a typical heart rate pattern recorded during a hot flash event occurring during sleep;

FIG. 1B shows a typical heart rate pattern recorded during a hot flash event occurring during wakefulness;

FIG. 1C shows a typical heart rate pattern recorded during short awakenings during sleep;

FIG. 1D shows a typical heart rate pattern recorded during long awakenings during sleep;

FIG. 2 is a schematic diagram of a system for detecting the occurrence of hot flashes in an individual and initiating therapeutic measures based thereon according to an exemplary embodiment of the disclosed concept; and

FIG. 3 is a block diagram showing the internal components of a wearable sensor unit according to one non-limiting exemplary embodiment of the disclosed concept;

FIG. 4 is a flowchart showing a method of detecting the occurrence of hot flashes in an individual and initiating therapeutic measures based thereon according to an exemplary embodiment of the disclosed concept;

FIG. 5 is a schematic diagram of a system for detecting the occurrence of hot flashes in an individual and initiating therapeutic measures based thereon according to an alternative exemplary embodiment of the disclosed concept;

FIG. 6 is a schematic diagram of a system for detecting the occurrence of hot flashes in an individual and initiating therapeutic measures based thereon according to a further alternative exemplary embodiment of the disclosed concept;

FIG. 7 shows a typical set of weight values that correspond to the typical heart rate pattern observed before and after a hot flash event that may be used in one exemplary implementation of the disclosed concept; and

FIGS. 8A and 8B illustrate operation of one exemplary implementation of the disclosed concept wherein template matching is employed.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “number” shall mean one or an integer greater than one (i.e., a plurality).

As used herein, the term “controller” shall mean a number of programmable analog and/or digital devices (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

As used herein, the term “deep learning neural network” shall mean an artificial neural network with multiple hidden layers between the input and output layers that determines the correct mathematical manipulation (linear or non-linear) to turn the input into the output by moving through the layers and calculating the probability of each output.

As used herein, the term “hidden layer” shall mean a neural network layer of one or more neurons whose output is connected to the inputs of other neurons and that, as a result, is not visible as a network output.

As used herein, the term “recurrent neural network” shall mean a class of artificial neural network where connections between nodes form a directed graph along a temporal sequence and that therefore allows the network to exhibit temporal dynamic behavior.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

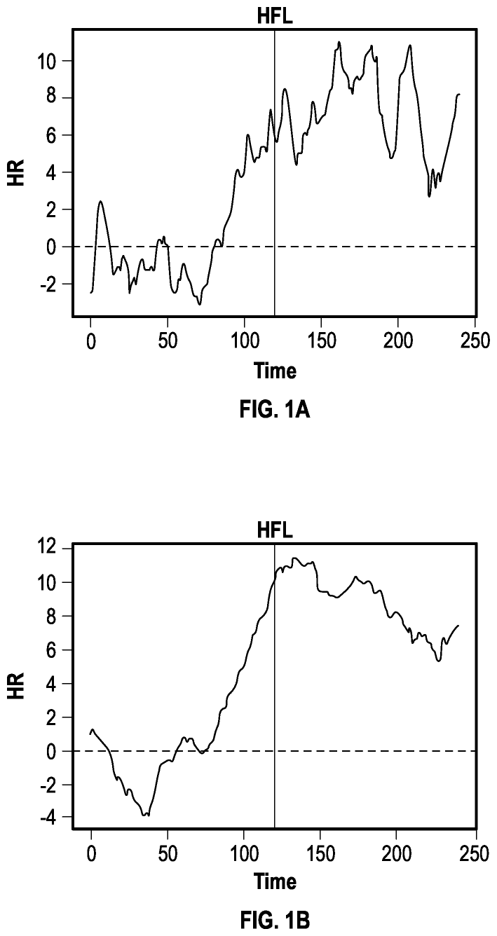

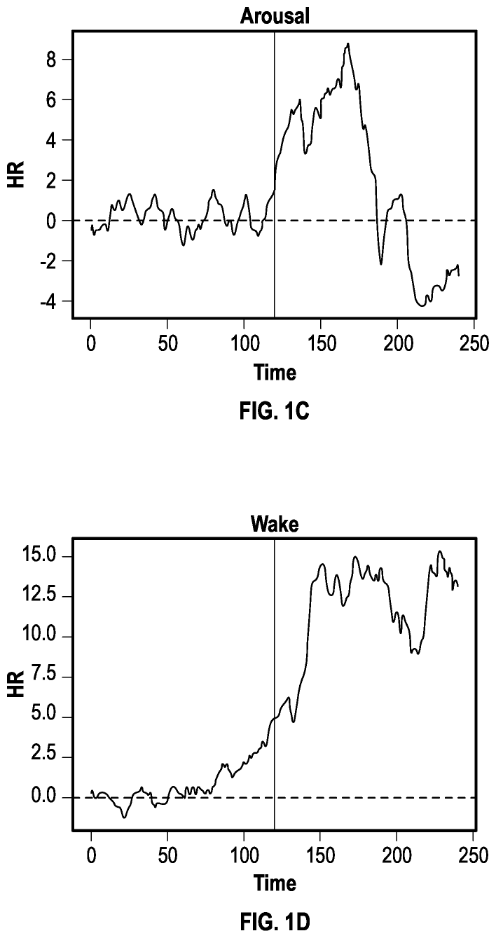

The disclosed concept originates from the original insight that the time course of heart rate (as measured with a device such as, without limitation, an electrocardiogram (ECG or EKG) sensor or a photoplethysmograph (PPG) sensor) before a hot flash event substantially differs from that of other types of arousals observed during sleep. More specifically, FIG. 1A shows a typical heart rate pattern recorded during a hot flash event occurring during sleep, FIG. 1B shows a typical heart rate pattern recorded during a hot flash event occurring during wakefulness (e.g., daytime), FIG. 1C shows a typical heart rate pattern recorded during short awakenings during sleep (due to non-hot flash arousals), and FIG. 1D shows a typical heart rate pattern recorded during long awakenings during sleep (due to non-hot flash arousals). The vertical line in each FIG. identifies the start time of each event within the temporal sequence. As seen in FIGS. 1A and 1B, heart rate measurements derived during hot flash events show a depression from baseline of at least a first amount (e.g., 4 beats per minute in the 2 minutes before the event) before the hot flash event followed by a rapid increase over baseline of at least a second amount (e.g., 8 beats per minute) during the event. Contrarily, as seen in FIGS. 1C and 1D, non-hot flash arousals do not show any heart rate drop from baseline prior to the event, and instead show an opposite heart rate trend, namely, a heart rate depression following a heart rate rise during the event. This indicates that it would be possible to discriminate those increases in heart rate occurring during sleep that are related to hot flash events as compared to non-hot flash events based on the time course of heart rate, as measured by a sensor such as a PPG or ECG sensor.

The disclosed concept, as described in greater detail herein in various particular exemplary embodiments, thus provides a method to automatically detect hot flash events for long-term monitoring of women affected by this menopause-related condition using heart rate data captured using a sensor such as a PPG or ECG sensor which is incorporated in a wearable device, such as a wrist watch, an in-ear device, a chest strap, or a patch. Heart rate measurements may this be carried out continuously during the day and during the night to detect hot flash onset and activate therapeutic measures to mitigate symptoms. As a result, the user will no longer suffer from awakenings due to hot flashes, and sleep quality will improve.

As described in greater detail herein in connection with various particular exemplary embodiments, the disclosed concept includes the following steps. First, cardiac activity is monitored using a wearable sensor as described above, which confers high unobtrusiveness, a feature that is essential for convenient long-term use. This includes detection of heartbeats and calculation of heart rate data from the wearable-device signal waveform. Next, a sequence of heart rate values is defined over a certain period of time (e.g. 4 minutes). Then, the mean heart rate is determined in an initial period (e.g., the first 10 seconds) of the sequence. The heart rate values in the sequence are then normalized, for example by subtracting the determined mean heart rate from each value. The sequence of normalized heart rate values is then processed using a computational model, such as, without limitation, a recurrent neural network, a dense layer of neurons, or a filter that contains information on the temporal heart rate progression before a hot flash event, to determine the probability that a hot flash event is occurring. Finally, a binary decision between hot flash or non-hot flash event is made by processing and thresholding the sequence of likelihood/probability values. If a hot flash is determined to be occurring, therapeutic mitigation steps may then be automatically initiated. In addition, or alternatively, a record tracking hot flash events may be modified in order to provide objective indication of occurrences. This is particularly interesting given that at night, events may be forgotten in recall diaries due to sleep. As a result, the record of events over time may be used to observe trends and manage the condition.

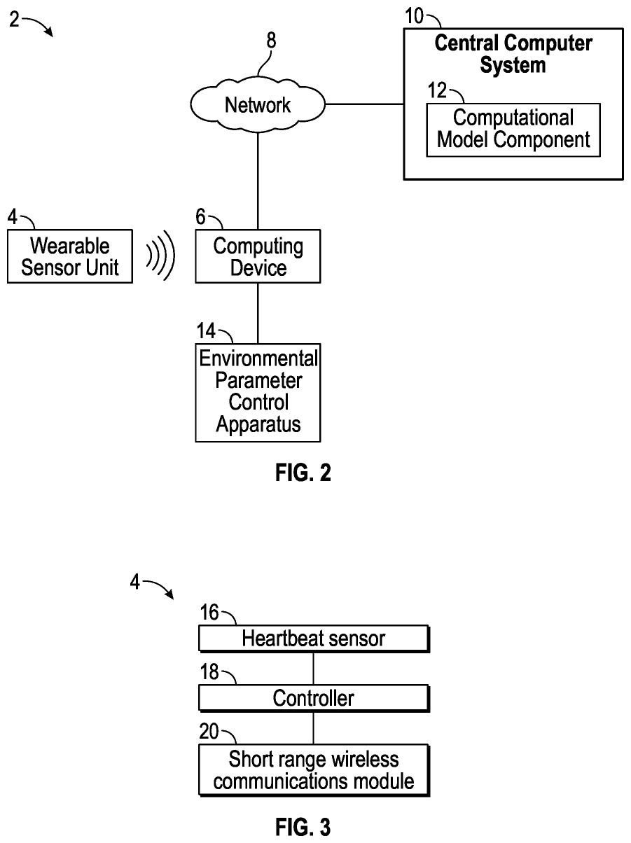

FIG. 2 is a schematic diagram of a system 2 for detecting the occurrence of a hot flash in an individual and initiating therapeutic measures based thereon according to an exemplary embodiment of the disclosed concept. As seen in FIG. 2, system 2 comprises a plurality of components including a wearable sensor unit 4, a computing device 6 in proximity to and in electronic communication with wearable sensor unit 4, a network 8, a central computer system 10 including a computational model component 12, and an environmental parameter control apparatus 14. Each of these components is described in detail below. As seen in FIG. 2, computing device 6, central computer system 10, and environmental parameter control apparatus 14 are all in electronic communication with network 8 to facilitate operation of system 2 as described herein. Furthermore, while in the illustrated exemplary embodiment computational model component 12 resides in the “cloud”, it will be understood that is may also implemented locally on a computing device such as a PC.

Wearable sensor unit 4 is structured and configured to be worn by an individual to be monitored. FIG. 3 is a block diagram showing the internal components of wearable sensor unit 4 according to one non-limiting exemplary embodiment. The exemplary wearable sensor unit 4 includes a heartbeat sensor 16 structured and configured to generate data representing deserted heartbeats (i.e., heartbeat data) for the individual wearing wearable sensor unit 4. In the exemplary embodiment, heartbeat sensor 16 is a PPG sensor or an ECG sensor (e.g., 1 to 12 leads), although it will be appreciated that other types of heart parameter sensors may also be employed within the scope of the disclosed concept. For example, and without limitation, heartbeat sensor 16 may also be a ballistocardiographic sensor, such as sensors measuring body movement and vibration of the chest due to the heart beating (e.g., an accelerometer positioned at the chest to measure heartbeat data).

Wearable sensor unit 4 further includes a controller 18 coupled to receive the outputs of heartbeat sensor 16 and, in the non-limiting exemplary embodiment, is structured and configured to determine heart rate and heart rate sequence data therefrom as described herein. Finally, wearable sensor unit 4 includes a short-range wireless communications module 20 that is structured and configured to enable wearable sensor unit 4 to communicate with computing device 6 over a short-range wireless network. Short-range wireless communications module 20 may be, for example and without limitation, a WiFi module, a Bluetooth® module, a ZigBee module, an IEEE802.15.4 module, or any other suitable short-range wireless communications module that provides compatible communications capabilities.

Referring again to FIG. 2, in the exemplary embodiment, computing device 6 may be, for example and without limitation, a smartphone, a tablet PC, a laptop computer, or some other computing device. Computing device 6 may also be a non-portable computing device such as a desktop PC. computing device 6 is structured to be able to communicate wirelessly with wearable sensor unit 4 over a short-range wireless network as described above. In addition, computing device 6 is structured and configured to be able to communicate with network 8 by way of a wired or wireless connection. In the exemplary embodiment, computing device 6 stores and implements a software application (e.g., a web/mobile app) that allows it to collect and transmit data as described herein.

Network 8 may be, for example, the Internet, one or more private communications networks, or any combination thereof. As employed herein, the term “communications network” shall expressly include, but not be limited by, any local area network (LAN), wide area network (WAN), intranet, extranet, global communication network, the Internet, and/or wireless communication network. Preferably, the wired and/or wireless connections to network 8 described herein are secure (e.g., in the form of an encrypted virtual private network).

Central computer system 10 comprises any suitable processing or computing system having a computing device and one or more memory components for data storage (e.g., a controller), such as, without limitation, one or more PCs or server computers. As seen in FIG. 10, central computer system 10 houses and implements a computational model component 12 for processing data received by central computer system 10 as described herein. More specifically, central computer system 10 has stored therein a number of routines that are executable by controller and that implement (by way of computer/processor executable instructions stored on a tangible medium) at least one embodiment of computational model component 12 as described herein. Computational model component 12 may be, for example and without limitation, a template matching system or an artificial intelligence system, such as a deep learning neural network that comprises a recurrent neural network or a dense layer of artificial neurons. In the case where computational model component 12 comprises an artificial intelligence system, the disclosed concept contemplates that such an artificial intelligence system will be trained and tested using certain training heart rate data to be able to assess, on a go forward basis, the probability of a hot flash occurring based on received heart rate data. In particular, as described in more detail herein, such an artificial intelligence based system would be trained to examine temporal changes in heart rate data (determined from heartbeat data) in order to determine from such data the probability that the individual is experiencing a hot flash event.

Environmental parameter control apparatus 14 is a device that is associated with the location, such as a home, hospital or nursing facility, in which the individual wearing wearable sensor unit 4 resides. Environmental parameter control apparatus 14 is structured and configured to implement therapeutic measures (e.g., temperature changes) when hot flashes are detected as described herein, and may be, for example and without limitation, a computer controlled HVAC system, cooling blanket or water cooled cooling system. The exemplary environmental parameter control apparatus 14 includes a controller that is structured and configured to receive and implement commands sent by computing device 6.

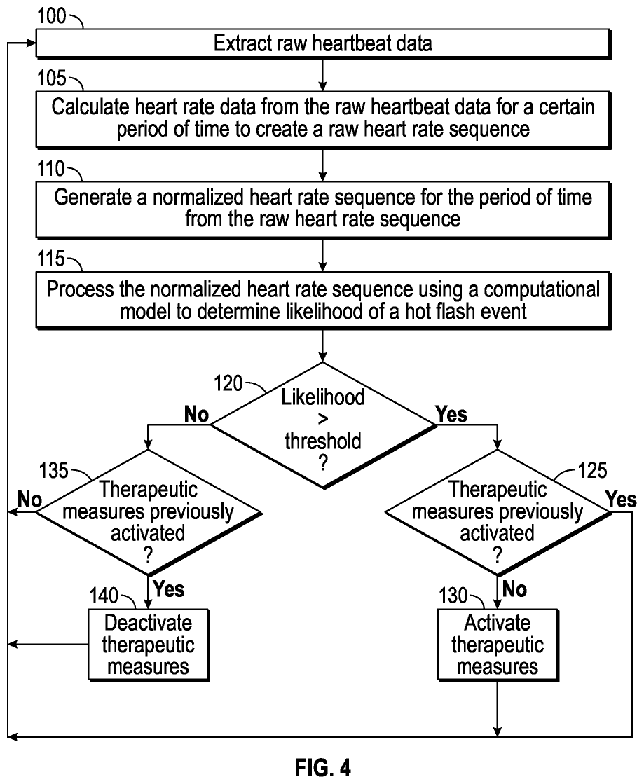

FIG. 4 is a flowchart showing a method of detecting the occurrence of hot flashes in an individual and initiating therapeutic measures based thereon according to an exemplary embodiment of the disclosed concept. The method of FIG. 4 is, in the illustrated exemplary embodiment, implemented by system 2 of FIG. 2. The method begins at step 100, wherein wearable sensor unit 4 extracts raw heartbeat data from the individual wearing wearable sensor unit 4. As discussed elsewhere herein, in the exemplary embodiment, wearable sensor unit 4 employs heartbeat sensor 16 for such purpose. In addition, in the exemplary embodiment, heartbeat data comprises data identifying RR intervals as detected by heartbeat sensor 16, and may be in the form of ECG QRS waveform data or PPG pulse wave data. Next, at step 105, controller 18 of wearable sensor unit 4 calculates heart rate data from the raw heartbeat data for a certain period of time (e.g., four minutes) to create a raw heart rate sequence (comprising a plurality of raw heart rate values). Then, at step 110, controller 18 generates a normalized heart rate sequence for the period of time from the raw heart rate sequence. In the exemplary embodiment, the normalized heart rate sequence is generated by determining the mean heart rate during an initial period of the sequence, such as the first ten seconds. Then, the heart rate values in the sequence are normalized by subtracting the calculated mean heart rate value from each heart rate value in the sequence. It will be appreciated, however, that this method of normalization is meant to be exemplary only, and that other methods of normalizing the data are contemplated within the scope of the disclosed concept. For example, other normalization techniques may include applying standard scaling or min-max scaling to achieve a similar result.

Next, wearable sensor unit 4 transmits the normalized heart rate sequence data to computing device 6. In the exemplary embodiment, this is done wirelessly by way of short-range wireless communications module 20 of wearable sensor unit 4. It will be appreciated, however, that other methods of communicating such data are also possible. Computing device 6 then communicates the normalized heart rate sequence data to central computer system 10 through network 8. Next, at step 115, the normalized heart rate sequence data is processed by computational model component 12 of central computer system 10 in order to determine the likelihood or probability that the heart rate sequence data is indicative of an actual hot flash event. As discussed elsewhere herein, computational model component 12 may be implemented in a number of different alternative exemplary manners, several of which are discussed in detail herein.

Next, at step 120, a determination is made as to whether the determined probability or likelihood is greater than some predetermined threshold. If the answer at step 120 is yes, then the method proceeds to step 125, wherein a determination is made as to whether any therapeutic measures had been previously activated. If the answer is yes, then the method returns to step 100. However, if the answer at step 125 is no, then the method proceeds to step 130. At step 130, central computer system 10 take steps to cause therapeutic measures to be activated. In particular, in the exemplary embodiment, central computer system 10 generates one or more control signals which are transmitted through network 8 to computing device 6 and then to environmental parameter control apparatus 14 which cause environmental parameter control apparatus 14 to initiate certain therapeutic measures for the detected hot flash. For example, if environmental parameter control apparatus 14 is an HVAC system, it will be caused to lower the temperature in the individual's present location in order to cool the individual. Alternatively, if environmental parameter control apparatus 14 is a device such as a cooling blanket or a water cooled system (e.g., a water cooled bed), the device will be activated in order to lower the temperature of the individual. The method then returns to step 100. If the answer at step 120 is no, however, then the method, rather than proceeding to step 125, proceeds to step 135. At step 135, a determination is made as to whether therapeutic measures were previously activated. If the answer is no, then the method proceeds to step 100. If, however, the answer at step 135 is yes, then the method proceeds to step 140, wherein the previously activated therapeutic measures are deactivated by central control system 10 (by way of appropriate command signals), as they are no longer needed. The method then returns to step 100.

System 2 of FIG. 2 and the method shown in FIG. 4 and just described thus provide an automated mechanism for detecting the occurrence of hot flashes by monitoring for temporal trends in heart rate that are indicative of hot flashes. Upon detection of a hot flash, the mechanism initiates therapeutic measures for mitigating the effects of the detected hot flash.

While system 2 and the method shown in FIG. 4 as described above provide one exemplary implementation of the disclosed concept, it will be appreciated that alternatives thereto are contemplated within the scope of the disclosed concept. For example, while the method as described includes the calculation of the raw heart rate sequence data and the normalized heart rate sequence data by wearable sensor unit 4, it will be appreciated that such steps may be performed by other components of system 2, such as, for example, computing device 6 or central computer system 10. Once such steps are performed, the normalized heart rate sequence data may then be processed by computational model component 12 as discussed herein.

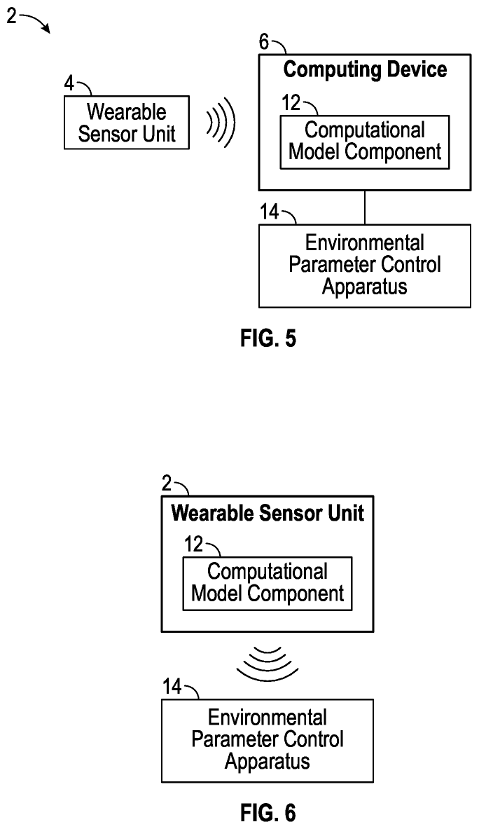

Still further alternatives are possible. For example, FIG. 5 shows an alternative system 2′ that is similar to system 2, except that rather than having computational model component 12 residing in and being implemented by central computer system 10, it is instead resident in and implemented by computing device 6 such that the processing may be done locally in computing device 6. In this example, computing device 6 issues the commands to control environmental parameter control apparatus 14 as warranted. As another example, FIG. 6 shows a further alternative system 2″ that is similar to systems 2 and 2′, except that rather than having computational model component 12 residing in and being implemented by central computer system 10 or computing device 6, it is instead resident in and implemented by controller 18 of wearable sensor unit 4 such that the processing may be done locally in wearable sensor unit 4. In this example, wearable sensor unit 4 issues the commands (wirelessly by way of short-range wireless communications module 20) to control environmental parameter control apparatus 14 as warranted.

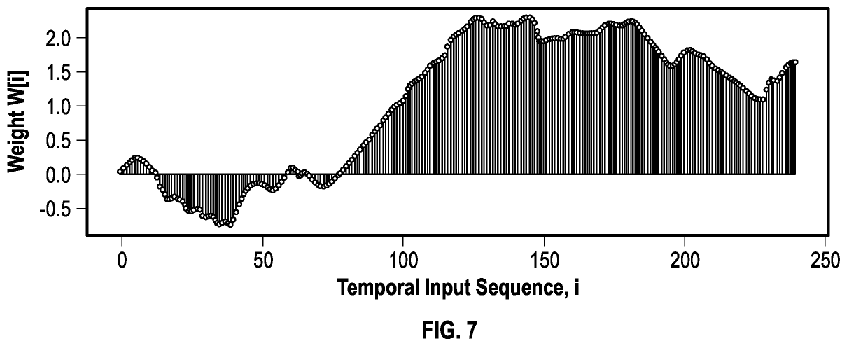

In one particular exemplary embodiment, computational model component 12 (wherever it resides) is structured and configured to implement a template matching approach. In one specific implementation, the template matching approach includes multiplying the input heart rate sequence (e.g., the normalized heart rate sequence data) with a template of weights (w[i]) that describe the likely heart rate pattern during a hot flash event as described elsewhere herein. FIG. 7 shows a typical set of weights having values that correspond to the typical heart rate pattern observed before and after a hot flash event. According to the equation below:

$${M}_{HFL} = \frac{1}{Ni} {\sum }_{i = 0}^{Ni} w [ i ] \times HR [ i ] ,$$

the level of agreement between the input heart rate pattern (HR[i] of length Ni) and the hot flash pattern (w[i] of length Ni) is defined by MHFL (matching value between hot flash template and heart rate sequence). After computing the MHFL describing the level of agreement between the heart rate sequence (e.g., the normalized heart rate sequence described herein) and the typical hot flash heart rate pattern, a post-processing algorithm can be used to sharpen the identification of the starting moment of the hot flash event. It has been observed that the standard deviation over 20 seconds (Nw=20) of the differential of the matching value (MHFL) as output by the template matching function can be used to identify hot flash events when a PHFL (probability of a hot flash event) value exceeds a certain threshold. In the exemplary embodiment, PHFL is determined according to the following equations:

$${P}_{HFL} [ i ] = \sqrt{\frac{1}{Nw} {\sum }_{i = 0}^{Nw} {[ {dM}_{HFL} [ i ] - \frac{1}{Nw} {\sum }_{j = 0}^{Nw} {dM}_{HFL} [ j ] ]}^{2}}$$

$${P}_{HFL} = {\sum }_{j = 0}^{Nj} A [ j ] \times {\sum }_{j = 0}^{Ni} ( w [ i , j ] \times HR [ i ] ) \vert .$$

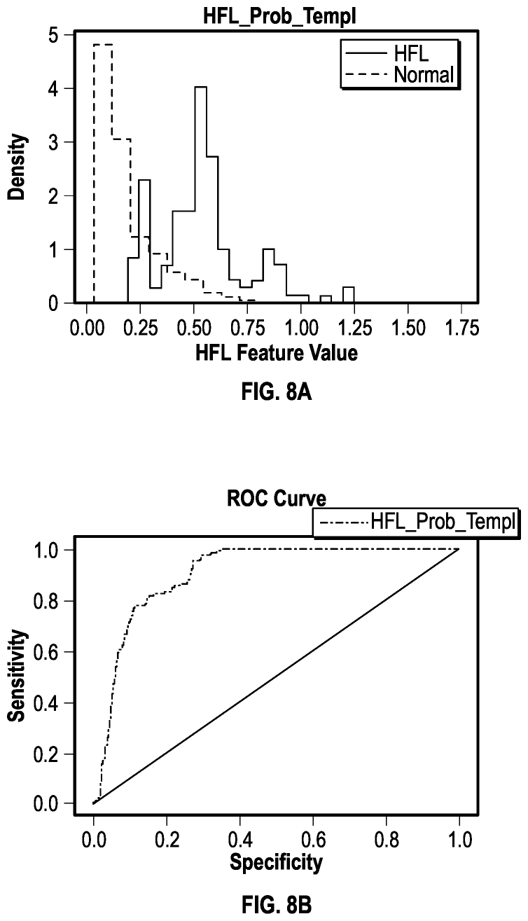

FIGS. 8A and 8B show the distribution of PHFL values in a 240 second sequence surrounding a hot flash event vs sequences measured during the entire night in a training dataset. It can be seen that PHFL increases in correspondence of hot flash events. At the same time, FIGS. 8A and 8B show that by thresholding the PHFL value, it is possible to achieve high accuracy for hot flash detection during the night as indicated by the area under the ROC curve, which is largely above random chance (black diagonal line).

In an alternative embodiment, computational model component 12, instead of measuring agreement between a sequence and a pre-defined template as just described, could be one or more dense layers of artificial neurons, in which the weights applied to the input heart rate values could produce a likelihood of hot flash (PHFL) based on the sum of the activation functions of each node in the dense layer according to the following:

PHFL=Σj:0 . . . Nj(A[j]×(Σi:0 . . . Ni(w[i,j]×HR[i]))),

where A[j] is the activation function (e.g. rectified linear, logit, etc) of each j-node in the dense layer (with Nj=240 nodes), w[i, j] are the values of the weights used to modulate the values in the input layer towards the j-node, and HR[i] represent the value at the i-position in the input layer which is the size of the entire hear rate segment (Ni=240 input nodes: HR/sec×60 sec/min×4 min). The sum of the output from the processing nodes in the dense layer could be fed in some other dense layer or used as output to describe a value proportionate to the probability of hot flash given the HR[i] input.

In another alternative embodiment, the HR[i] input sequence could be processed using a deep learning neural network/algorithm such as a recurrent neural network (long-short term memory layer, gru, etc). This type of neural network layer is particularly indicated to discover the peculiar temporal pattern in the input data to represent an output value such as a probability of hot flash events.

As still another alternative embodiment, other vital signs (respiration rate, blood pressure, PPG waveform, body temperature) for which the time course during hot flash events differs from non-hot flash related arousals and awakenings could also be captured by wearable sensor unit 4 (equipped with one or more appropriate sensors) and used in addition to heart rate to reliably detect hot flash event onset.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of detecting an occurrence of a hot flash in an individual, comprising:
obtaining heart rate sequence data for the individual for a predetermined period of time, wherein the heart rate sequence data is based on heartbeat data of the individual that is detected by a sensor unit worn by the individual;
providing the heart rate sequence data to a computational model component, wherein the computational model component is structured and configured to examine the heart rate sequence data over time to determine a probability that the individual is experiencing a hot flash based on monitoring the heart rate sequence data for a pattern wherein heart rate decreases below a baseline range and then increases above the baseline range; and
analyzing the heart rate sequence data in the computational model component to determine the probability.

2. The method according to claim 1, further comprising assessing the determined probability to determine whether a hot flash is indicated, and if a hot flash is determined to be indicated by the determined probability, causing an environmental parameter control apparatus associated with the individual to initiate therapeutic measures for the hot flash.

3. The method according to claim 1, further comprising assessing the determined probability to determine whether a hot flash is indicated, and if a hot flash is determined to be indicated by the determined probability, storing a record of the indicated hot flash.

4. The method according to claim 1, wherein in the pattern, heart rate decreases below the baseline range by at least a first magnitude then increases above the baseline range by at least a second magnitude that is larger than the first magnitude.

5. The method according to claim 4, wherein in the pattern, heart rate decreases below the baseline range by at least the first magnitude within a certain restricted period of time before heart rate then increases above the baseline range by at least the second magnitude.

6. The method according to claim 1, wherein the assessing the determined probability to determine whether a hot flash is indicated comprises determining whether the probability is above a threshold value.

7. The method according to claim 1, wherein the heart rate sequence data is normalized heart rate sequence data generated from raw heart rate sequence data that is based on the heartbeat data of the individual that is detected by the sensor unit.

8. The method according to claim 7, wherein the raw heart rate sequence data comprises a sequence of heart rate values, and wherein the normalized heart rate sequence data is generated by determining a mean in the heart rate values for an initial period of the sequence and then subtracting the mean from the heart rate values of the sequence.

9. The method according to claim 1, wherein the sensor unit comprises at least one of a PPG sensor, an ECG sensor or an accelerometer for generating the heartbeat data.

10. The method according to claim 1, wherein the computational model component employs a template matching approach for determining the probability that the individual is experiencing a hot flash based on monitoring the heart rate sequence data for the pattern.

11. The method according to claim 10, wherein the template matching approach includes determining a matching value by multiplying the heart rate sequence data with a template of weights that describe the pattern.

12. The method according to claim 11, wherein the template matching approach includes using a standard deviation of a differential of the matching value to identify hot flash events when a probability value exceeds a certain threshold.

13. The method according to claim 1, wherein the computational model component employs a dense layer of artificial neurons for determining the probability that the individual is experiencing a hot flash based on monitoring the heart rate sequence data for the pattern.

14. The method according to claim 13, wherein each node in the dense layer has an activation function, and wherein weights are applied to heart rate sequence data to produce a likelihood of hot flash based on a sum of the activation functions of each node in the dense layer.

15. The method according to claim 1, wherein the computational model component employs a deep learning neural network for determining the probability that the individual is experiencing a hot flash based on monitoring the heart rate sequence data for the pattern.

16. The method according to claim 1, wherein the environmental parameter control apparatus is one of an HVAC system, a cooling blanket and a water cooled cooling system.

17. A computer program product, comprising a non-transitory computer usable medium having a computer readable program code embodied therein, the computer readable program code being adapted and configured to be executed to implement a method of detecting the occurrence of a hot flash as recited in claim 1.

18. An apparatus for detecting an occurrence of a hot flash in an individual, comprising:
a controller including a computational model component, wherein the computational model component is structured and configured to receive heart rate sequence data that is based on heartbeat data of the individual that is detected by a sensor worn by the individual and examine the heart rate sequence data over time to determine a probability that the individual is experiencing a hot flash based on monitoring the heart rate sequence data for a pattern wherein heart rate decreases below a baseline range and then increases above the baseline range.

19. The apparatus according to claim 18, wherein the controller is structured and configured to assess the determined probability to determine whether a hot flash is indicated, and if a hot flash is determined to be indicated by the determined probability, cause an environmental parameter control apparatus associated with the individual to initiate therapeutic measures for the hot flash.

20. The apparatus according to claim 18, wherein in the pattern, heart rate decreases below the baseline range by at least a first magnitude then increases above the baseline range by at least a second magnitude that is larger than the first magnitude.

21. The apparatus according to claim 18, wherein the heart rate sequence data is normalized heart rate sequence data generated from raw heart rate sequence data that is based on the heartbeat data of the individual that is detected by the sensor unit.

22. The apparatus according to claim 21, wherein the raw heart rate sequence data comprises a sequence of heart rate values, and wherein the normalized heart rate sequence data is generated by determining a mean in the heart rate values for an initial period of the sequence and then subtracting the mean from the heart rate values of the sequence.

23. The apparatus according to claim 18, wherein the computational model component employs a template matching approach for determining the probability that the individual is experiencing a hot flash based on monitoring the heart rate sequence data for the pattern.

24. The apparatus according to claim 23, wherein the template matching approach includes determining a matching value by multiplying the heart rate sequence data with a template of weights that describe the pattern.

25. The apparatus according to claim 24, wherein the template matching approach includes using a standard deviation of a differential of the matching value to identify hot flash events when a probability value exceeds a certain threshold.

26. The apparatus according to claim 18, wherein the computational model component employs a dense layer of artificial neurons for determining the probability that the individual is experiencing a hot flash based on monitoring the heart rate sequence data for the pattern.

27. The apparatus according to claim 26, wherein each node in the dense layer has an activation function, and wherein weights are applied to heart rate sequence data to produce a likelihood of hot flash based on a sum of the activation functions of each node in the dense layer.

28. The apparatus according to claim 18, wherein the computational model component employs a deep learning neural network for determining the probability that the individual is experiencing a hot flash based on monitoring the heart rate sequence data for the pattern.

29. The apparatus according to claim 18, wherein the sensor is part of a wearable sensor unit structured to be worn by the individual and the controller resides within a computing device located separately from the wearable sensor unit.

30. The apparatus according to claim 18, wherein the controller and the sensor are part of a wearable sensor unit structured to be worn by the individual.
